(19) United States
(12) Patent Application Publication
(10) Pub. No.: US 2017/0267967 A1
(43) Pub. Date: Sep. 21, 2017

(54) AUTOMATIC METHOD FOR MONITORING CELL CULTURE GROWTH

(71) Applicant: Fraunhofer-Gesallschaft Zur Förderung Der Angewandten Forschung E.V., München (DE)

(72) Inventors: Marc DRÖGE, Hamm (DE); Christian MÜLLER, Eitorf (DE); Anja LINNEMANN, Sankt Augustin (DE); Harald Peter MATHIS, Bonn (DE); Stefan BORBE, Sankt Augustin (DE); Torsten HEUP, Bergheim-Fliesteden (DE); Andreas PIPPOW, Köln (DE)

(21) Appl. No.: 15/516,284

(22) PCT Filed: May 11, 2015
(86) PCT No.: PCT/EP2015/060297

§ 371 Date: Mar. 31, 2017

(30) Foreign Application Priority Data
Oct. 7, 2014 (DE) 10 2014 220 306.6

(51) Int. Cl. C12M 1/36; C12M 1/34; C12Q 1/18

(52) U.S. Cl. CPC C12M 41/48; C12Q 1/18; C12M 41/46

(57) ABSTRACT

In the automated method according to the invention for monitoring cell culture growth, in particular bacterial growth, a receiving dish (24) with a nutrient medium (38) is provided onto and/or into which is applied a cell culture, in particular a sample of human or animal tissue, such as blood, to which bacteria have been added and which also contains one or a plurality of reagents, in particular antibiotics. A microscope (10) having a camera (18) and an image acquisition plane adapted to be moved along the optical axis (20) is provided, and the receiving dish (24) is brought into the microscope (10) for monitoring a potential growth of the cell culture in the nutrient medium (38). At predetermined time intervals, in particular in the single-digit minute range, an image of the nutrient medium (38) is acquired using the camera (18) of the microscope (10) by moving the image acquisition plane along the optical axis (20) through the nutrient medium (38) by means of the movable slide (14) and/or the acquisition optic unit (16), one image per image acquisition plane is acquired, and from the group of acquired images the highest-contrast image of the nutrient medium (38) or an image of the nutrient medium (38) with a contrast sufficient for the subsequent automatic further processing of the image is automatically selected and stored, where required, by means of an image evaluation software. By means of the image evaluation software the size of the area occupied by the cell culture is automatically determined on the basis of the selected image. On the basis of the sizes of the areas occupied by the cell culture of the respective selected images it is determined whether the cell culture is growing or not.

The invention relates to an automatic method in particular for real-time tracking of cell culture growth and in particular of bacterial growth.

There are various fields of application where it is of interest to be able to monitor cell culture growth in real time, if possible. One example of this is the determination of resistance to/efficacy of antibiotics.

The conventional method for determining resistance to antibiotics and/or for determining the efficacy of antibiotics is based on a macroscopic approach. This method takes a relatively long time since evaluations of the growth can be effected only when bacterial colonies are visible to the naked eye. This may be achieved within a period of 6 to 24 hours, which is not tolerable in time-critical cases.

In WO-A-2010/129532 a method and a device for rapidly providing evidence of resistant bacteria are described. Here, immobilized bacterial cells in a culture medium with or without antibiotics are monitored by means of a microscope.

In addition, in Marlene Fredborg et al., “Real-Time Optical Antimicrobial Susceptibility Testing”, Journal of Clinical Microbiology, volume 51, number 7, 2013, pp. 2047-2053, a device for image processing for tests for resistance to antibiotics is described.

An alternative semi-automatic method for the rapid susceptibility testing of microorganisms exists under the trade name VITEK®2. Here, a transmission optics is employed. The amount of light shining through a sample allows conclusions to be drawn with regard to the cell culture growth. The stronger the growth, the less intensive is the transmitted light. Unfortunately this method, too, reliably provides first results only after a few hours at the earliest.

The two aforementioned methods are not suitable for determining the effect of antibiotics on individual cells but require a plurality of cells to be monitored and/or the turbidity of a bacterial suspension.

It is an object of the invention to suggest an automated method for monitoring cell culture growth, in particular bacterial growth, wherein it is possible to considerably more rapidly evaluate the growth than in conventional methods.

For achieving this object, the invention suggests an automated method for monitoring cell culture growth, in particular bacterial growth, wherein in the method
a receiving dish with a nutrient medium is provided onto and/or into which is applied a cell culture, in particular a sample of human or animal tissue, such as blood, to which bacteria have been added and which also contains one or a plurality of reagents, in particular antibiotics,
a microscope having an optical axis and including a camera and a slide for the receiving dish adapted to be automatically moved along the optical axis and/or an acquisition optic unit adapted to be automatically moved along the optical axis are provided,
the receiving dish is brought into the microscope for monitoring a potential growth of the cell culture in the nutrient medium,
at predetermined time intervals, in particular in the single-digit minute range, an image of the nutrient medium is acquired using the camera of the microscope by moving the image acquisition plane along the optical axis through the nutrient medium by means of the movable slide and/or the acquisition optic unit of the microscope, one image per image acquisition plane is acquired, and from the group of acquired images the highest-contrast image of the nutrient medium or an image of the nutrient medium with a contrast sufficient for the subsequent automatic further processing of the image is automatically selected and stored, where required, by means of an image evaluation software,
by means of the image evaluation software the size of the area occupied by the cell culture is automatically determined on the basis of the selected image, and
on the basis of the sizes of the areas occupied by the cell culture of the respective selected images it is determined whether the cell culture is growing or not.

In the method according to the invention, an image processing software is employed for evaluating images of the cell culture automatically acquired at time intervals. A nutrient medium containing the cell culture is applied in particular into a miniaturized receiving dish. The nutrient medium contains one or more reagents, in particular antibiotics. The cell culture consists in particular of bacteria which are to be tested for their resistance to antibiotics or by means of which the efficacy of an antibiotic is to be tested.

The automatic image acquisition carried out at time intervals is effected with the aid of a (in particular reflected-light or transmitted-light) microscope having a camera and an acquisition optic unit as well as a slide for the receiving dish, which are adapted to be moved relative to each other along the optical axis, in particular in very small steps. This allows to always automatically select the image acquisition plane such that images with as high a contrast as possible of the cell culture can be acquired. For this purpose, the image acquisition plane is moved through the nutrient medium by moving the acquisition optic unit and/or the slide along the optical axis, wherein at the different image acquisition planes a respective image is acquired. By a subsequent or simultaneously performed image processing the highest-contrast image is determined and selected. Alternatively, it may be sufficient to select one of the highest-contrast images from the group of the respective acquired images as long as the contrast is sufficiently large to allow the subsequent image evaluation to be carried out. Such types of image processing are e. g. employed in autofocus cameras and can thus be used in the invention.

For the purpose of selecting the highest-contrast image or the image with a contrast sufficient for the subsequent further processing of the image either an image can first be acquired and stored for each approached image acquisition plane and selection can be subsequently carried out, or the image acquired first can be stored as the image determined as having the highest contrast so far and each further acquired image is compared to the stored image, and when this image has a higher contrast that the stored image, it is stored as the image determined as having the highest contrast so far.

By means of another image processing software module the respective selected image can be automatically examined with respect to the size of the area occupied by the cell culture. Thus the growth of the cell culture can be monitored quasi in real time. One example of a software suitable for this purpose is described in Andreas Pippow, Stefan Borbe, Sebastian Rose, Stefan Precht, Thomas Berlage, “Zellteilungsdauer im High-Content-Screening bestimmen”, Laborwelt, No. 1/2012, pp. 33 and 34, and Thomas Berlage, Andreas Pippow, “Neues Potential für die Pharmaforschung”, GIT Labor-Fachzeitschrift, October 2013, pp. 630 to 635.

An essential feature of the invention is the possibility of the automatic image acquisition with an optimum contrast, which requires movability of the acquisition optic unit and/or the slide of the microscope as in the case of the autofocus function. For the purpose of acquisition of high-contrast images the image acquisition plane must be repositioned from time to time for just the reason that the nutrient medium may evaporate in the course of time and thus the nutrient medium level in the receiving dish changes.

Appropriately, the microscope comprises a slide for the receiving dish adapted to be moved along the optical axis, in particular in very small steps of some tens of μm. It should also be contemplated to move the acquisition optic unit of the microscope along the optical axis, however, this requires a considerably larger effort than moving the slide. Here, a drive unit would be required for allowing for movement in appropriately very small steps.

According to a further appropriate aspect of the invention, it may be provided that on the basis of the sizes of the areas occupied by the cell culture of the respective selected images it is determined whether the cell culture is growing, and if so, at which growth rate.

As has already been stated above, it is to be proceeded from the assumption that at the respective points in time when the next image with as high a contrast as possible is to be acquired, the image acquisition plane has changed with respect to the respective former position. In order to save time, instead of the respective travel of the image acquisition plane through the nutrient medium for obtaining an image with as high a contrast as possible, it may be advantageous that the region within which the image acquisition plane travels in the nutrient medium for determining the image of the nutrient medium with the highest contrast or for determining an image of the nutrient medium with a contrast sufficient for determining the size of the area of the cell culture is limited to the region around the position of the image acquisition plane of the temporally last image or one of the temporally previous images. Here, it is proceeded from the assumption that the image acquisition plane where the supposedly sharpest image can be acquired hardly changes from one acquisition time to the next acquisition time. However, the respective complete travel of the image acquisition plane through the nutrient medium may be required at the individual image acquisition times because the autofocus unit has been misadjusted due to external influences, e. g. thermal influences or vibrations, since the last image acquisition time.

As has already been stated above, it is appropriate for various reasons to use as small a receiving dish as possible. One possibility is to use a miniaturized receiving dish which comprises two sheets having a frame arranged therebetween. The two sheets are glass slides, for example. The frame surrounds a receiving space which is closed towards the sides by the frame and towards the top and the bottom by the two sheets. Into this receiving space the nutrient medium (e. g. agar) is applied to which the cell cultures to be monitored and one of more reagents are added.

As has been stated above, the subject matter of the present invention is an automated method for real-time tracking of bacterial growth and/or of cell culture growth and/or of growth of microorganisms. The method is based on the use of a microscope which, at defined time intervals and supported by an autofocus unit, captures pictures of in particular miniaturized growth areas (nutrient medium) on which cell cultures grow. The pictures and/or images are subsequently preprocessed and analyzed by an image processing software to prepare a growth curve or another type of representation of the growth of the cell culture on the basis of the size of the area of the images which have been acquired and evaluated at the respective points in time.

In the automated method according to the invention for monitoring cell culture growth, in particular bacterial growth, thus a receiving dish with a nutrient medium is provided onto and/or into which is applied a cell culture, in particular a sample of human or animal tissue, such as blood, to which bacteria have been added and which also contains one or a plurality of reagents, in particular antibiotics. A microscope having a camera and an image acquisition plane adapted to be moved along the optical axis is provided and the receiving dish is brought into the microscope for monitoring a potential growth of the cell culture in the nutrient medium. At predetermined time intervals, in particular in the single-digit minute range, an image of the nutrient medium is acquired using the camera of the microscope by moving the image acquisition plane along the optical axis through the receiving dish and thus through the nutrient medium, one image per image acquisition plane is acquired, and from the group of acquired images the highest-contrast image of the nutrient medium or an image of the nutrient medium with a contrast sufficient for the subsequent automatic further processing of the image by means of an image evaluation software is automatically selected and stored, where required. By means of the image evaluation software the size of the area occupied by the cell culture is automatically determined on the basis of the selected image. On the basis of the sizes of the areas occupied by the cell culture of the respective selected images it is determined whether the cell culture is growing or not.

The method according to the invention allows for the real-time tracking of bacterial growth on the plane of bacterial colonies as well as on a cellular plane, which means that, on the one hand, a growth curve can rapidly be derived based on monitoring the increase in the growth areas and, on the other hand, additionally the cell division of each individual can be tracked. This allows for acceleration as compared with conventional resistance tests, a new approach for the antibiotics research, for example, as well as the automated tracking of bacterial growth/bacterial division over short and long periods.

The effects and advantages of the invention can be summarized as follows:
Due to employment of optimized miniaturized growth areas an automated microscopic tracking of the growth on a cellular plane is allowed for. In addition, the miniaturization of the growth areas allows for saving reagents and sample material, which offers an economic advantage as compared with the conventional method.
Due to use of an autofocus unit of the microscope an automated capturing of pictures/images at certain time intervals is possible. Without the autofocus unit a manual refocusing prior to each capturing of a new picture would be necessary, which would be contrary to automation.
Due to preparation of growth curves of the area where e. g. bacteria grow the determination of the minimum inhibitory concentration (MIC) of antibiotics can be significantly accelerated, namely be effected in 2 to 3 hours instead of 5 to 24 hours as in prior art.

Besides the preparation of a growth curve on the basis of an area determination based on a foreground/background recognition, the invention is further based on the use of an autofocus function based on a contrast intensity and the use of in particular miniaturized growth areas (on the basis of agar or on the basis of another nutrient medium, for example).

Cases of application of the method according to the invention are
monitoring of (bacterial) cell divisions,
determination of resistance to antibiotics,
monitoring of the influence of various substances on the bacterial growth and/or the cell divisions, and
monitoring of the influence of combinatory antibiotics administration on the bacterial growth and/or the cell divisions.

An image processing software for automatically determining the size of selected regions of an image, such as the area occupied by the cell culture in the present case, is generally known. One example of this is the software with the trade name ZETA developed by the applicant (see the two aforementioned non-patent citations).

Hereunder the invention is described in detail with reference to the drawing in which

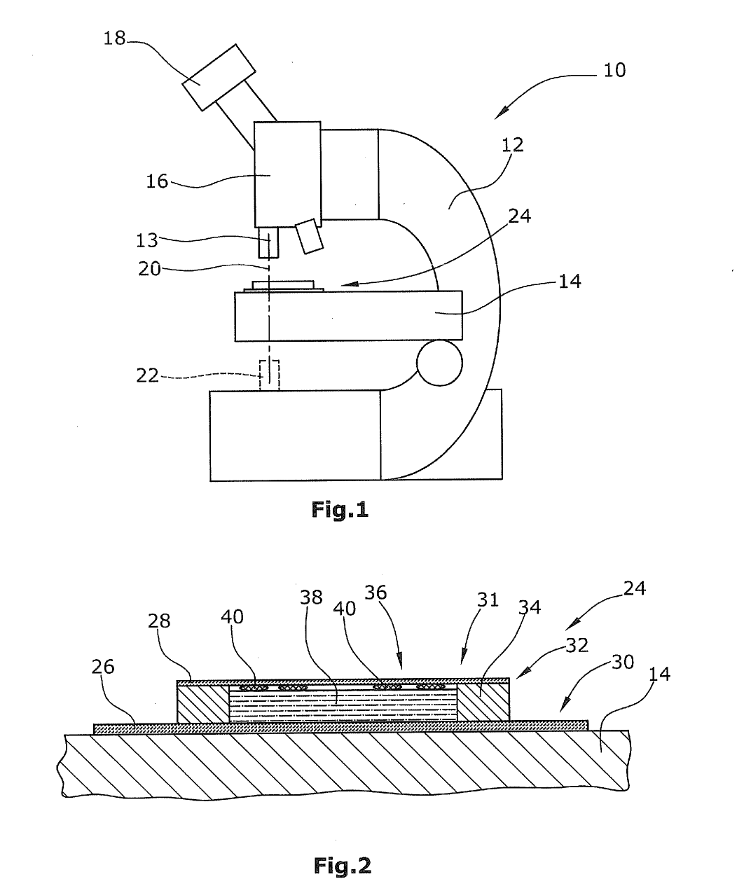

FIG. 1 schematically shows a representation of a reflected-light microscope to be used according to the invention,

FIG. 2 shows a magnified view of the object slide arrangement having a miniaturized receiving dish,

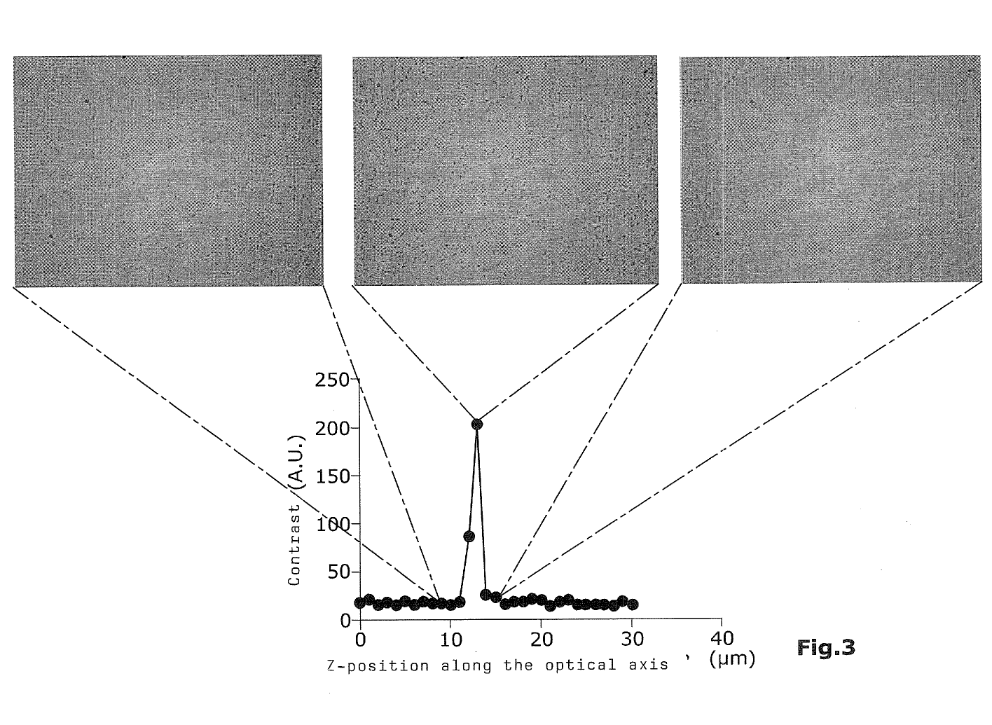

FIG. 3 schematically shows the functional principle for automatically determining the highest-contrast image,

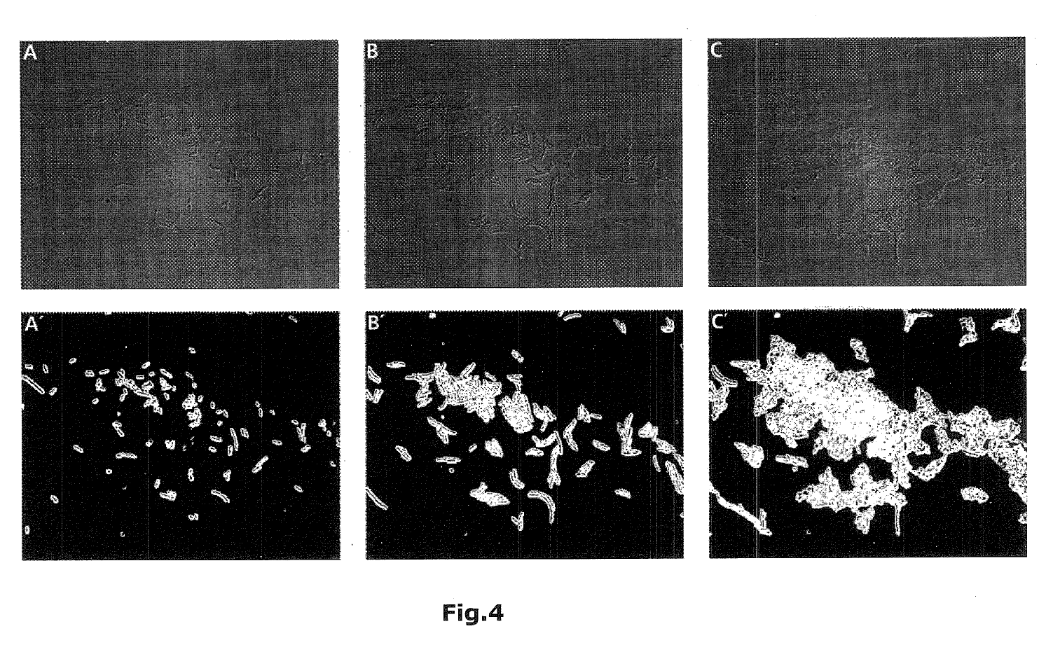

FIG. 4 shows examples of pictures of the growing cell culture at various points in time A, B and C and the processed images corresponding to this pictures for foreground/background recognition for the purpose of automated determination of cell culture areas, and

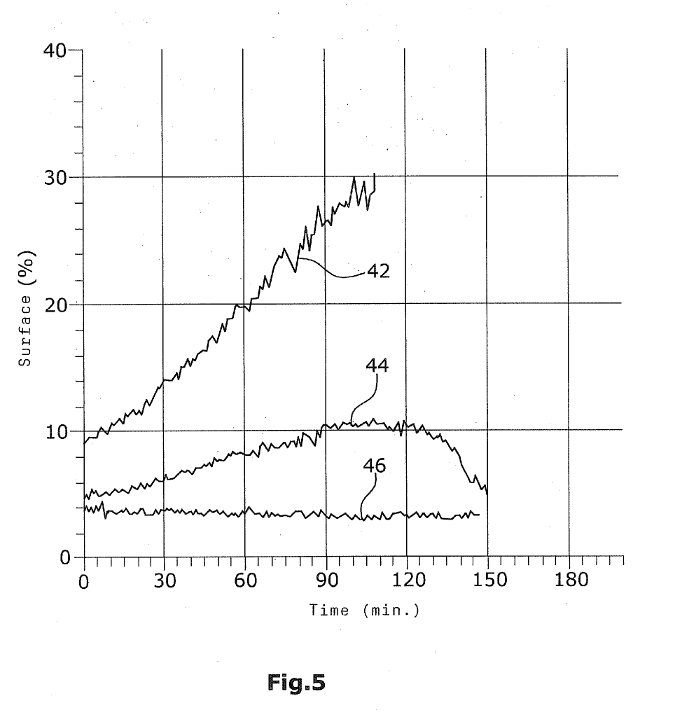

FIG. 5 shows three examples of growth curves prepared according to the invention.

In FIG. 1 an exemplary embodiment of a reflected-light microscope 10 is shown which comprises a microscope body 12 having a reflected-light illumination unit 13 and a slide 14 (movable Z stage) adapted to be moved in particular in very small steps along the optical axis of the microscope. The microscope body 12 further includes an acquisition optic unit 16 and a camera 18. The optical axis is indicated at 20.

Alternatively to the reflected-light microscope the invention also allows for employing a transmitted-light microscope. The position of the transmitted-light illumination unit to be employed in this case is shown as a dashed line at 22.

On the slide 14 a receiving dish 24 is located which is shown in FIG. 2 at a larger scale. The receiving dish 24 comprises a bottom object slide 26 and a cover slip 28, in particular configured as two sheets or panes 30, 31 between which a frame 34 in particular configured as a double-faced adhesive film 32 is arranged. This frame 34 defines in its interior a receiving space 36 for a nutrient medium 38 (which is agar, for example) on which bacteria 40 grow. The nutrient medium is provided with a reagent, such as an antibiotic. The bacteria 40 may e. g. be part of a blood sample which is to be examined.

FIG. 3 illustrates the functional principle of the automated determination of the highest-contrast image which is acquired at a certain point in time after the start of the bacterial growth. By means of the slide 14 adapted to be moved in very small steps the image acquisition plane is moved through the receiving dish 24. In doing so, one image is acquired per step. The contrast intensity in different Z-positions is queried. In FIG. 3 this is shown using the example of a bacterial culture of Staphylococcus aureus. The graph of FIG. 3 shows the contrast intensity (in A. U.) as a function of the Z-position (in μm). Three images showing the associated contrast intensities are exemplarily shown.

FIG. 4 shows examples of the foreground/background recognition by means of the aforementioned ZETA software of the applicant using the example of an Escherichia Coli (E. coli) growth analysis. The upper row (A-C) shows the original images, wherein A represents the start of a time series, B has been acquired after approximately one hour and C has been acquired after 6 hours. The second lower row (A′-C′) shows the corresponding foreground/background recognition as black-and-white representations.

Finally, FIG. 5 shows three examples of bacterial growth curves determined according to the invention of a test for resistance to antibiotics of E. coli for LB medium (see curve 42), for ampicillin (see curve 44) and for kanamycin (see curve 46).

1. An automated method for monitoring cell culture growth, in particular bacterial growth, wherein in the method comprises:
providing a receiving dish with a nutrient medium onto and/or into which is applied a cell culture, in particular a sample of human or animal tissue, such as blood, to which bacteria have been added and which also contains one or a plurality of reagents, in particular antibiotics,
providing a microscope having an optical axis and including a camera and a slide for said receiving dish adapted to be automatically moved along said optical axis and/or an acquisition optic unit adapted to be automatically moved along said optical axis,
wherein said receiving dish is brought into said microscope for monitoring a potential growth of the cell culture in said nutrient medium,
acquiring at predetermined time intervals, in particular in the single-digit minute range, an image of said nutrient medium is acquired using said camera of said microscope by moving the image acquisition plane along said optical axis through said nutrient medium by means of at least one of said movable slide and said acquisition optic unit of said microscope, one image per image acquisition plane is acquired, and from the group of acquired images the highest-contrast image of said nutrient medium or an image of said nutrient medium with a contrast sufficient for the subsequent automatic further processing of the image is automatically selected and stored, where required, by means of an image evaluation software,
wherein by means of the image evaluation software the size of the area occupied by the cell culture is automatically determined on the basis of the selected image, and
wherein on the basis of the sizes of the areas occupied by the cell culture of the respective selected images it is determined whether the cell culture is growing or not.

2. The method according to claim 1, wherein on the basis of the sizes of the areas occupied by the cell culture of the respective selected images it is determined whether the cell culture is growing, and if so, at which growth rate.

3. The method according to claim 1, wherein the step of the image acquisition plane traveling through the receiving dish and the nutrient medium is effected by moving optical elements of the acquisition optic unit of the microscope along the optical axis and/or by moving the slide comprising said receiving dish along said optical axis of said microscope.

4. The method according to claim 1, wherein the region within which the image acquisition plane travels in the nutrient medium for determining the image of said nutrient medium with the highest contrast or for determining an image of said nutrient medium with a contrast sufficient for determining the size of the area of the cell culture is limited to the region around the position of the image acquisition plane of the temporally last image or one of the temporally previous images.

5. The method according to claim 1, wherein the receiving dish comprises two sheets, in particular an object slide and a cover slip having a frame arranged therebetween, wherein the region surrounded by said frame defines the receiving space of said receiving dish.

6. The method according to claim 1, wherein on the basis of the sizes of the areas occupied by the cell culture of the respective selected images a growth curve is prepared.

7. The method according to claim 1, wherein for selecting the highest-contrast image or for selecting the image with a contrast sufficient for the subsequent further processing of the image either an image is first acquired and stored for each approached image acquisition plane and the selection is subsequently carried out, or the image acquired first is stored as the image determined as having the highest contrast so far and each further acquired image is compared to the stored image, and when this image has a higher contrast than the stored image, it is stored as the image determined as having the highest contrast so far.

8. The method according to claim 1, wherein the microscope is a reflected-light or a transmitted-light microscope.
